(19) United States
(12) Patent Application Publication
(10) Pub. No.: US 2015/0179079 A1
(43) Pub. Date: Jun. 25, 2015

(54) MOBILE DEVICES AS NEURAL SENSORS FOR IMPROVED HEALTH OUTCOMES AND EFFICACY OF CARE

(71) Applicant: New Technologies & Associates, Inc., Boston, MA (US)

(72) Inventors: Raphael A. Rodriguez, JR., Quincy, MA (US); Rowland T. Graus, Reston, VA (US)

(21) Appl. No.: 14/582,670

(22) Filed: Dec. 24, 2014

Related U.S. Application Data
(60) Provisional application No. 61/920,594, filed on Dec. 24, 2013.

(51) Int. Cl. G09B 5/00; A61B 5/00; A61B 5/01; A61B 5/16; G09B 19/00; A61B 5/11

(52) U.S. Cl. CPC G09B 5/00; G09B 19/00; A61B 5/1124; A61B 5/01; A61B 5/162; A61B 5/7264

(57) ABSTRACT

A system and method is provided for real time monitoring a patient's cognitive and motor response to a stimulus, the system comprising: A mobile or tablet device; a user interface disposed on the mobile device; sensors monitoring user interaction with the mobile device and capturing kinesthetic and cognitive data; a processor comparing the kinesthetic and cognitive data and comparing the data to a baseline, and identifying relative improvement and impairment of cognition and motor skills from the comparison.

### RELATED APPLICATIONS

This application claims the benefit of U.S. Provisional Applications No. 61/920,594, filed Dec. 24, 2013. This application is herein incorporated by reference in its entirety for all purposes.

### FIELD OF THE INVENTION

The invention relates to patient monitoring systems, and more particularly, to a patient monitoring system deployed on a mobile or tablet device.

### BACKGROUND OF THE INVENTION

With the advent of mobile devices, tablets and smartphones there is an opportunity to utilize these devices as a mobile sensor. Today's devices have sensors such as dual phones, dual microphones, accelerometers, GPS and radio, magnetometer, ambient light detection, proximity and gyroscopes. Tomorrow's devices will add additional sensor capabilities enabling further improvements to the invention for sensor-based collection. Mobile devices, tablets and smartphones use touchscreens and flat glass surfaces to capture many of these sensor capabilities. The touchscreen gives attributes such as 2D coordinates, area, angle and orientation of the contacts and pressure sensor. The touchscreen data can capture sequence, X/Y/Z coordinates, timestamps for dwell time, flight time, and key to key as well as touch to touch times in addition to size, pressure, active and reactive movement shift, touch exchange, globularity, intensity and orientation data.

In today's healthcare environment there is a drive to lower healthcare costs, increase quality of care and to increase the efficacy of care and measured quality outcomes of specific health treatments and gold standard protocols of care. Once patients are not in the physical custody of healthcare providers and clinicians there is currently little treatment oversight beyond having healthcare providers and clinicians call over the telephone or use electronic communications such as instant messaging, email or other electronic means. While there has been a push toward the development of wearable devices dedicated to addressing specific health concerns or conditions (e.g., Fitbit, Neumitra), these solutions create manufacturing and distribution challenges. Additionally, they are limited in functionality by their dedicated hardware design. Furthermore, dedicated devices may not already be owned by the patient, requiring them to acquire such a device at additional cost, availability and inconvenience. As a result, the market still lacks an affordable and effective way to measure a patient for their mental or physical state without a face-to-face meeting or at best a phone based interview.

What is needed, therefore, are techniques for monitoring patient cognitive and motor skills in real time outside of a clinical setting.

### SUMMARY OF THE INVENTION

One embodiment of the present invention provides a system for real time monitoring a patient's cognitive and motor response to a stimulus, the system comprising: A mobile or tablet device; a user interface disposed on the mobile device; sensors monitoring user interaction with the mobile device and capturing kinesthetic and cognitive data; a processor comparing the kinesthetic and cognitive data and comparing the data to a baseline, and identifying relative improvement and impairment of cognition and motor skills from the comparison.

Another embodiment provides such a system wherein the processor comprises a decision engine and an admin engine.

A further embodiment provides such a system wherein the processor is configured to use both semantic and neural network analytics and processing.

Still another embodiment provides such a system wherein the system is configured to use and access at least one analytic locally over a WAN/LAN or in the cloud or across multiple clouds selected from the group of Big Data analytics, visual analytics, and predictive analytics for processing, data discovery or analysis.

A still further embodiment provides such a system wherein the user interface displays a prompt to the user eliciting a response from the user.

Yet another embodiment provides such a system wherein the kinesthetic and cognitive data is dwell time.

A yet further embodiment provides such a system wherein the kinesthetic and cognitive data is location of touch event on the device.

Even another embodiment provides such a system wherein the kinesthetic and cognitive data is active shift.

An even further embodiment provides such a system wherein the kinesthetic and cognitive data is reactive shift.

Even yet another embodiment provides such a system further comprising at least one additional sensor selected from the group of sensors consisting of temperature sensors, magnetometers, chemical sensors, conductivity sensors, and touch characteristic sensors.

An even yet further embodiment provides such a system further comprising a user identity validation system.

Still yet another embodiment provides such a system wherein the kinesthetic and cognitive data comprise additional data selected from the group of data consisting of intensity, exchange, x-y-z force, x-y-z motion, order, and flight.

One embodiment of the present invention provides a method for the real time monitoring a patient's cognitive and motor response to a stimulus, the method comprising: collecting user kinesthetic and cognitive data from user interaction with a mobile device; comparing with a processor user kinesthetic and cognitive data from user interaction with the mobile device with baseline kinesthetic data; identifying diagnostically significant deviations from the baseline kinesthetic and baseline cognitive data; classifying diagnostically significant deviations associated with associated cognitive symptoms; assessing cognitive symptoms based on known diagnosis; and determining the relative improvement or impairment based on assessment of the symptoms.

Another embodiment provides such a method wherein the processor comprises a decision engine and an admin engine.

A further embodiment provides such a method wherein the processor is configured to use both semantic and neural network analytics and processing.

Yet another embodiment provides such a method wherein the kinesthetic and cognitive data comprise at least one data selected from the group of data consisting of order, dwell, flight, location, exchange, intensity, active shift, reactive shift, x-y-z force, and x-y-z motion.

A yet further embodiment provides such a method wherein the baseline kinesthetic and cognitive data are past data of the patient.

Still another embodiment provides such a method wherein the baseline kinesthetic and cognitive data are aggregated data of a population of patients.

A still further embodiment provides such a method further comprising validating the patient's identity.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a flow chart illustrating a method for testing a user's cognitive and motor performance on a mobile device with real-time assessment configured in accordance with one embodiment of the present invention.

FIG. 2 is a block diagram illustrating a system for testing a user's cognitive and motor performance on a mobile device with real-time assessment configured in accordance with one embodiment of the present invention.

Exhibit A is a summary of one embodiment of the present invention and an explanation of its applications; this Exhibit is intended as an integral and indivisible part of this provisional application.

### DETAILED DESCRIPTION

The ability to capture rich physical usage and interaction data allows one to employ artificial intelligence capabilities, such as using a neural and semantic network approach, to algorithmically create and measure a cognitive and neural mind state of a user. Combining these measurements with prior usage patterns as well as data generated by a population of users with similar conditions or characteristics will enable more effective healthcare diagnoses and treatment pathways.

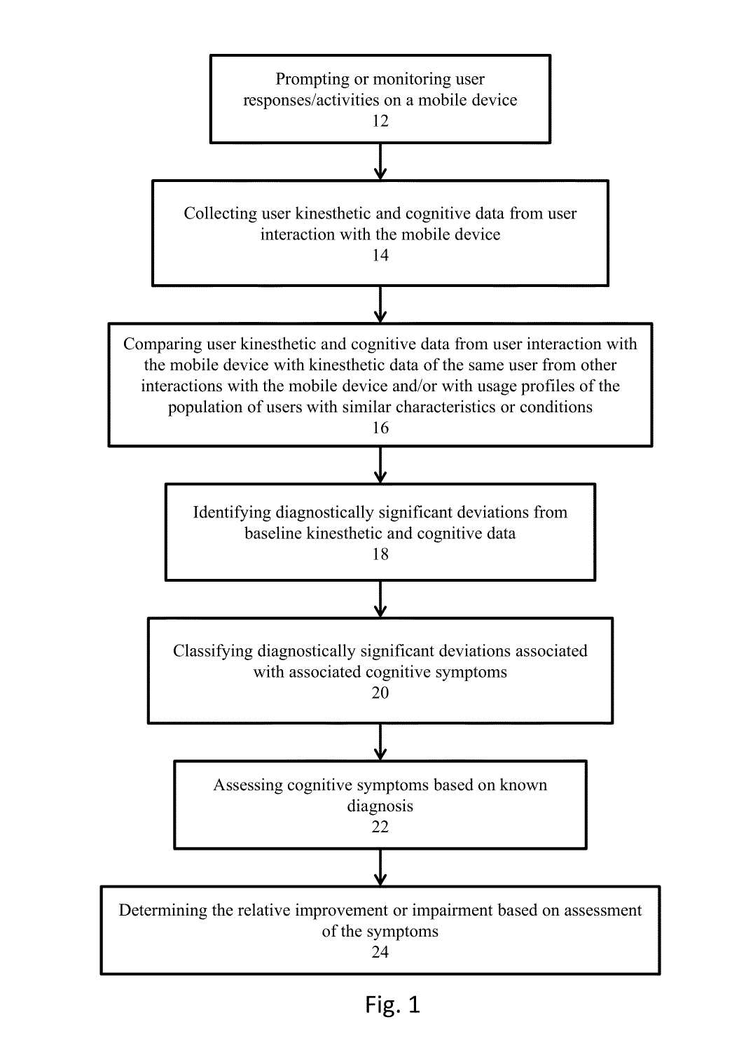

In one embodiment of the present invention, as illustrated in the flow chart of FIG. 1, a system and method for the monitoring of a patient. The system utilizes a lock screen or other User Interface (UI) requiring input by the patient of a user specific piece of information. Prompting user responses on a mobile device 12. One skilled in that art will appreciate that the action of prompting may be a specific prompt or may be inherent in other functions of the mobile device such as text messaging systems, games or other apps. All prompts either inherent or overt present the user with an opportunity to interact with the device. Prompts may take the form of written or spoken questions, unique pictorial prompts, gaming stimulus, or other stimuli. Information in such a response would be information that requires the user to respond, not from rote memory but from with instinctual or automatic response based on neural pathways. The type of interaction required would be tailored to specific user characteristics or conditions. Indeed, it is within the scope of this invention to passively monitor user interactions with the device and utilize responses made to other inquiries in lieu of test specific prompts. This allows the system to collect user kinesthetic and cognitive data from user interaction with the mobile device 14. Kinesthetic and cognitive data includes user reaction time and the sensory-motor data measured across a range of sensors on the mobile device. Real-time information is obtained by measuring artificial intelligence features such as time between touches of strokes and the duration of the stroke or touch itself, direction of movement, time to initiate the response, mobile device or smartphone movement, hold angle, physical touch intensity, and touch timing of said user as well as prompting said user to respond to highly personal questions or unique pictorial prompts to distinguish an increase of cognitive brain synapse response time or to detect a decrease or increase of mental cognition, motor control and executive control. Tasks testing manual dexterity, response, concentration, visual acuity, and motor control including the execution of specific movements. Such tasks may take the form of games, mobile alarms preset for different time of day or other tasks requiring sustained attention and cognitive performance, allowing for the measurement of prolonged neural cognition and a high degree of mental concentration.

Embodiments of the present invention may employ a variety of analytic techniques to obtain baselines and in analyzing the kinesthetic and cognitive data comparisons, including access at least one analytic locally over a WAN/LAN connection or accessed in the cloud or across multiple clouds via HTTP or HTTP/S and selected from the group of large data sets such as Big Data analytics, visual analytics, and predictive analytics for processing, data discovery or analysis

In one embodiment patient mobile enrollment and usage training is done by measuring one prior data collection set to establish a baseline. Every future mobile response is collected to enhance the training set of data for comparison to prior data. In other embodiments baselines may be established from data collected from patient populations. The system then compares kinesthetic data and cognitive data from the user interaction with the mobile device with kinesthetic data of the same user from other interactions with the mobile device and in those embodiments with a population based baseline, with the dataset of interactions from the population of users with similar characteristics or conditions. 16. The data collected from the mobile device can be compared to baseline data for the patient in real time, to allow the system to detect deviations from the baseline, while not all variations will be significant to the patient's decision, the system may identify diagnostically significant deviations from past kinesthetic and cognitive data 18. This may be determined either based on inputs from the clinician or other technician, by presets in the system, or by statistical analysis. In one embodiment, the data is compared to the patient or user's specific treatment protocol or standard of care. Such a system, allows for classifying diagnostically significant deviations associated with associated cognitive symptoms 20 thus assessing cognitive symptoms based on known diagnosis 22 or treatment protocol. The system of one embodiment of the present invention may also determine the relative improvement or impairment based on assessment of the symptoms 24. By enabling diagnostics and treatment assessments using commonly owned mobile devices, embodiments of the present invention radically reduce mobile treatment acquisition and distribution cost for healthcare providers and patients. Embodiments of the present invention will benefit from the natural evolution of sensor technology on state of the art mobile devices, which will ensure continued improvement in treatment protocols and efficacy.

Tasks asked of the user to elicit responses 12 may in some embodiments include cognitive or physical tasks. Such questions, suggestive reaction(s), visual, sound, vibrations, location aware, active and passive inputs would allow the system to directly and indirectly monitor cognitive and motor function in the patient, specifically using physical motor control and neural executive brain control to measure synapse memory functions and control. The tasks asked of the user to elicit responses 12 may in some embodiments come from one or more parts of the physical mobile sensors, embedded chipset software, device operating system (OS) or mobile application of the mobile device and tablet depending on where in the Open Systems Interconnection model (OSI) the desired functionality and sensory intercept is needed. The Open Systems Interconnection model (OSI) is a conceptual model that characterizes and standardizes the internal functions of a communication system by partitioning it into abstraction layers. The model is a product of the Open Systems Interconnection project at the International Organization for Standardization (ISO), maintained by the identification ISO/IEC 7498-1.

The model groups communication functions into seven logical layers. A layer serves the layer above it and is served by the layer below it. For example, a layer that provides error-free communications across a network provides the path needed by applications above it, while it calls the next lower layer to send and receive packets that make up the contents of that path. Two instances at one layer are connected by a horizontal connection on that layer. The specific task or tasks desired could come from the physical layer (layer 1) all the way up through the application layer (layer 7) within the OSI model.

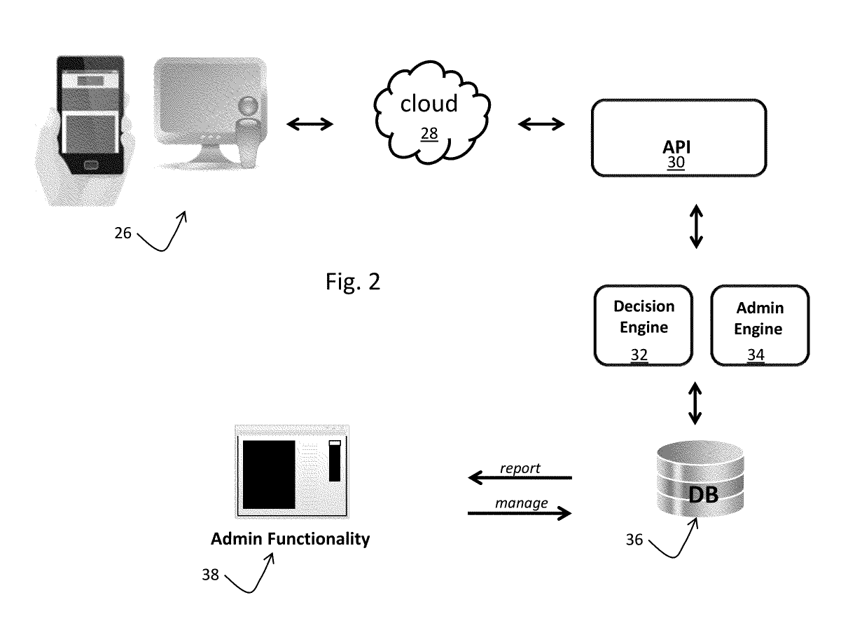

As illustrated in FIG. 2, the method may be practiced on a system comprising a user interface device 26, which detects the data and transmits it to the cloud 28. An application program interface (API) 30 retrieves the data from the cloud 28 and the data is provided to a decision engine 32 and an admin engine 34 which analysis the data using semantic and neural networking AI analytics and processing, which process. Analyzed data is output to a database 36 where it is stored and reported. An administrator 38 is provided which manages the system. The administrator 38 may be configured to receive reports of patient data or may provide

A mobile neural sensor and apparatus of one embodiment of the present invention may be used to sense, track and measure cognitive training-related improvements or degradation in real-time, including measures of fluid intelligence to immediately assess cognitive performance and executive motor control based on past performance and time-dependent decay principles measurements. Such an embodiment may sense, track and measure in real-time the cognitive performance and executive motor control of a user to determine the peak cognitive point based on the core body temperature (CBT) of a user based on the human circadian rhythms within a 24 hour daily human cycle to detect different cognitive capabilities and executive motor control by modulating time of day mobile sensing using a simple alarm or a series of alarms within the 24 hr cycle of testing.

Alternatively, one embodiment may provide a method and system to sense, track and measure in real-time the cognitive performance and executive motor control of a user by employing emotion and mood regulation strategies prior, during and after mobile neural sensing to foster positive emotion regulation and affective neurological functioning. In this embodiment the real-time measurement and subsequent treatment of patients with mental health issues such as anxiety, depression, and Schizophrenia, for example could be deployed.

In one embodiment, the mobile neural sensor and apparatus may be used to sense, track and measure in real-time the cognitive performance and executive motor control of a user across various mobile games designed to track and measure in real-time the core cognitive capacities, such as working memory, attention, speed of processing and fluid reasoning. In such an embodiment, game performance data could be combined with kinesthetic data created from the user's interaction with the mobile device to deepen the diagnostic capabilities compared to game performance analysis alone. This invention could also be combined with existing games on mobile devices, produced originally for other purposes, to enable cognitive data collection for healthcare purposes. This real-time measurement and subsequent cognitive training could be used to detect and measure the user's mental performance and ability to acquire new knowledge in order to affect positive cognition outcomes.

In one embodiment of the present invention a mobile neural sensor and apparatus may be used to sense, track and measure in real-time the cognitive performance and executive motor control of a user and the relationships between cognitive performance and lifestyle factors such as hours of sleep per night, alcohol intake, and physical exercise related to baseline performance on various cognitive tasks. In such an embodiment the real-time measurement and subsequent cognitive training help to detect and improve cognitive performances or to create and influence health efficacy and treatment protocols related to lifestyle factors, including average nightly sleep duration, weekly aerobic exercise amount, and daily alcohol intake.

The mobile neural sensor and apparatus of one embodiment of the present invention may be used to measure in real-time the touch responses of a user to a visual cue presented to said user. The distribution of previous behaviors of said user to the same visual cue can then be compared to said real-time capture to determine changes in cognitive performance and executive motor control in the user. The extent of changes can be compared to previous deviations from average behavior to determine the level of risk associated with the user's current performance.

The mobile neural sensor and apparatus of one embodiment of the present invention may be used to sense, track and measure in real-time the cognitive performance and executive motor control of a user for dexterity assessment. In this embodiment the real-time measurement could be used for patients who have executive motor control issues such as persons with multiple sclerosis, arthritis, muscular dystrophy or patients who are undergoing heart stroke rehabilitation to create and influence health efficacy and treatment protocols.

A similar system could be utilized to sense, track and measure in real-time the cognitive performance and executive motor control of a user for predictors of future heart related events such as acute heart attacks and stroke. Such a real-time measurement could be used for patients who are chosen for their pre-condition medical condition, pre or postoperative surgery or selected for treatment to influence health efficacy and treatment protocols

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A system for real time monitoring a patient's cognitive and motor response to a stimulus, the system comprising:
A mobile or tablet device;
a user interface disposed on the mobile device;
Sensors monitoring user interaction with said mobile device and capturing kinesthetic and cognitive data;
A processor comparing said kinesthetic and cognitive data and comparing said data to a baseline, and identifying relative improvement and impairment of cognition and motor skills from said comparison.

2. The system of claim 1 wherein said processor comprises a decision engine and an admin engine.

3. The system of claim 1 wherein said processor is configured to use both semantic and neural network analytics and processing.

4. The system of claim 1 wherein said system is configured to use at least one analytic selected from the group of Big Data analytics, visual analytics, and predictive analytics for processing

5. The system of claim 1 wherein said user interface displays a prompt to said user eliciting a response from said user.

6. The system of claim 1 wherein said kinesthetic and cognitive data is dwell time.

7. The system of claim 1 wherein said kinesthetic and cognitive data is location of touch event on said device.

8. The system of claim 1 wherein said kinesthetic and cognitive data is active shift.

9. The system of claim 1 wherein said kinesthetic and cognitive data is reactive shift.

10. The system of claim 1 further comprising at least one additional sensor selected from the group of sensors consisting of temperature sensors, magnetometers, chemical sensors, conductivity sensors, and touch characteristic sensors.

11. The system of claim 1 further comprising a user identity validation system.

12. The system of claim 1 wherein said kinesthetic and cognitive data comprise additional data selected from the group of data consisting of intensity, exchange, x-y-z force, x-y-z motion, order, and flight.

13. A method for the real time monitoring a patient's cognitive and motor response to a stimulus, the method comprising:
Collecting user kinesthetic and cognitive data from user interaction with a mobile device;
Comparing with a processor user kinesthetic and cognitive data from user interaction with said mobile device with baseline kinesthetic data;
Identifying diagnostically significant deviations from said baseline kinesthetic and baseline cognitive data;
Classifying diagnostically significant deviations associated with associated cognitive symptoms;
Assessing cognitive symptoms based on known diagnosis; and
Determining the relative improvement or impairment based on assessment of the symptoms.

14. The method of claim 13 wherein said processor comprises a decision engine and an admin engine.

15. The method of claim 13 wherein said processor is configured to use both semantic and neural network analytics and processing.

16. The method of claim 13 wherein said kinesthetic and cognitive data comprise at least one data selected from the group of data consisting of order, dwell, flight, location, exchange, intensity, active shift, reactive shift, x-y-z force, and x-y-z motion.

17. The method of claim 13 wherein said baseline kinesthetic and cognitive data are past data of said patient.

18. The method of claim 13 wherein said baseline kinesthetic and cognitive data are aggregated data of a population of patients.

19. The method of claim 13 further comprising validating said patient's identity.

20. The method of claim 13 further comprising prompting said patient to interact with said device.
